(19) United States
(12) Patent Application Publication
(10) Pub. No.: US 2020/0286627 A1
(43) Pub. Date: Sep. 10, 2020

(54) SYSTEMS AND METHODS FOR TREATMENT-EFFECT ANALYSIS

(71) Applicant: International Business Machines Corporation, Armonk, NY (US)

(72) Inventor: Hyuna Yang, San Jose, CA (US)

(21) Appl. No.: 16/295,796

(22) Filed: Mar. 7, 2019

(51) Int. Cl. G16H 50/70; G16H 10/60; G16H 50/20; G06F 17/27; G16H 70/00; G06N 20/20

(52) U.S. Cl. CPC G16H 50/70; G16H 10/60; G06N 20/20; G06F 17/27; G16H 70/00; G16H 50/20

(57) ABSTRACT

Systems and computer-implemented methods for analysis of medical literature to assemble synthetic cohorts are provided. In various embodiments, a plurality of literature sources relating to a medical condition are retrieved. The plurality of literature sources comprise published observational studies. A first plurality of features is extracted from the plurality of literature sources. A subpopulation is defined based on the first plurality of features. An effectiveness of a treatment within the subpopulation is predicted based on the plurality of features. A plurality of electronic medical records is retrieved. The effectiveness of the treatment is validated against the plurality of electronic medical records.

### BACKGROUND

Embodiments of the present disclosure relate to treatment-effect analysis, and more specifically, to systems and computer-implemented methods for analysis of medical literature to assemble synthetic cohorts.

### BRIEF SUMMARY

According to embodiments of the present disclosure, computer-implemented methods of and computer program products for treatment-effect analysis are provided. A plurality of literature sources relating to a medical condition are retrieved. The plurality of literature sources comprise published observational studies. A first plurality of features is extracted from the plurality of literature sources. A subpopulation is defined based on the first plurality of features. An effectiveness of a treatment within the subpopulation is predicted based on the plurality of features. A plurality of electronic medical records is retrieved. The effectiveness of the treatment is validated against the plurality of electronic medical records.

### BRIEF DESCRIPTION OF THE SEVERAL VIEWS OF THE DRAWINGS

FIG. 1 illustrates a process of evaluating treatment options according to the present disclosure.

FIG. 2 illustrates a method of treatment-effect analysis according to embodiments of the present disclosure.

FIG. 3 depicts a computing node according to an embodiment of the present disclosure.

### DETAILED DESCRIPTION

Understanding all available treatment options, treatment practices, and the effectiveness of each treatment in real world settings are important first steps of patient care. Many factors, such as patient demographics, disease stages, and complications have an impact on the best treatment option for a given disease/condition, and should be considered when selecting an appropriate treatment.

Various observational studies are available in the medical literature. Typically, such observational studies provide information regarding the effectiveness of a treatment within a particular cohort. Since different studies consider different factors (e.g. confounding variables, different time period, etc.) it is not always clear if a difference in results indicates conflicting study results or treatment-effect heterogeneity due to factors such as different population characteristics.

Occasionally, meta-analyses are published that obtain more robust results by combining several cohort-studies. In addition, review papers consolidate published results. However, there exists no solution capable of systematically evaluating findings appearing in the literature from different observational studies.

To address these and other shortcomings of alternative approaches, the present disclosure provides systems and methods for systematically evaluating many treatments under various conditions.

In various embodiments, study design components are extracted from publications. New cohorts are built for a plurality of study design combinations using secondary data such as EMR, registry, or claim data. Effectiveness of treatments are determined based on these cohorts. In various embodiments, confounders or inclusion/exclusion criteria information are extracted from publications. These may be used to define subpopulations for which effectiveness may be determined.

Secondary data may not fully replicate published studies, since they likely do not contain all features used in each publication. However, as an exploratory tool, the present disclosure evaluates effectiveness at various cohort and sub-populations. The results provide insights into personalized care (e.g., sub-populations sensitive to specific treatment), and can indicate a promising avenue for confirmatory study.

In various embodiments, retrospective observational studies are built based on design components specified by literature or user input. The effectiveness of treatment is evaluated with regard to features that secondary data can provide.

An observational study cohort is built from literature and user inputs. All study design components are collected (e.g., cohort definition, inclusion/exclusion criteria, primary/secondary endpoint, potential modifier/confounder, minimum sample size, study duration). Confounder and inclusion/exclusion criteria are further used to define sub-populations. A user can consolidate, remove, or add design components. For each treatment option, effectiveness is evaluated at every study design component combination. For each treatment option, effectiveness is evaluated at every study design component combination at each subpopulation. In various embodiment, to obtain a sufficiently large sample size, each sub-population is considered one at a time.

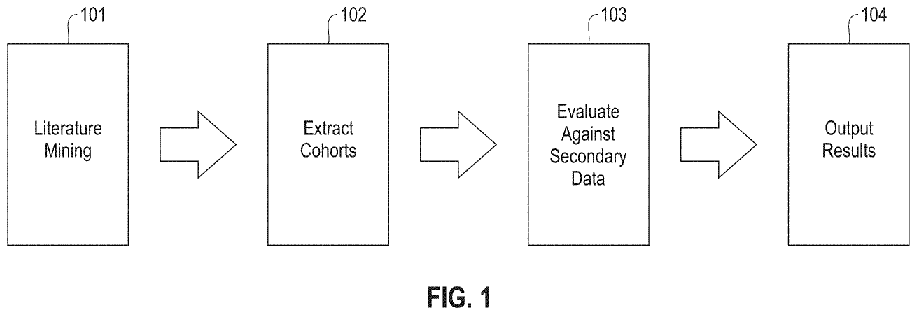

With reference now to FIG. 1, a process of evaluating treatment options is illustrated according to the present disclosure. At 101, literature mining is performed. A corpus of publications is searched for those that study the effectiveness of a given treatment of interest. From each study, a plurality of features is extracted. In some embodiments, the features include a disease or condition definition; an outcome; a standard of care; a treatment; confounding factors (factors, independently or with treatment, affecting outcomes); inclusion/exclusion criteria for the study; and effectiveness.

In some embodiments, the literature mining process includes extracting a key sentence from each study that includes the features of interest.

At 102, cohorts are extracted from the studies identified at 101. In particular, the features extracted from the literature are used to define a plurality of subpopulations. A user can consolidate, remove, or add design components. For each treatment option, effectiveness is evaluated at every study design component combination. For each treatment option, effectiveness is evaluated at every study design component combination at each subpopulation.

In some embodiments, a model is trained using the extracted features to predict treatment effectiveness for a given candidate treatment against a subpopulation. In some embodiments, the model is disease-specific. In some embodiments, the model comprises an SVM, CNN, LSTM. In some embodiments, the model is an ensemble model.

More particularly, in some embodiments, a feature vector is provided to a learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

At 103, the effectiveness of a given treatment of interest is evaluated based on features of secondary data. In this step, having identified a treatment of potential interest, secondary data, such as from an EMR or from an insurance claims database is used to support or refute the hypothetical treatment. Secondary data in this context refers to raw data from a plurality of patients, rather than data that results from a controlled study. This stage restricts the analysis to features in the secondary data.

An electronic health record (EHR), or electronic medical record (EMR), may refer to the systematized collection of patient and population electronically-stored health information in a digital format. These records can be shared across different health care settings and may extend beyond the information available in a PACS discussed above. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

In various embodiments, the user can consolidate or add study cohort components. The effectiveness of each treatment may then be compared with that of standard of care at each study cohort component combination (e.g., of the overall population). At each subpopulation and at each study cohort component combination, the effectiveness of each treatment may thereby be evaluated.

At 104, the resulting findings are summarized. In various embodiments, the validation results for each treatment hypothesis are summarized. In particular, the validation results are provided for the limited features available in the secondary data, thereby providing results from the subpopulations of interest.

The methods above allow researchers to evaluate treatment options by summarizing relevant publications, and evaluating treatment effectiveness using secondary data at various cohort setting. Researchers can thereby quickly understand all treatment options and their effectiveness for different customizable subpopulations.

When published results are contradictory, these methods of evaluation may bring additional insights as to what causes the contradiction.

Moreover, by extracting cohort study components, this information may be used to design further studies.

One alternative approach is to search observational studies targeting specific disease or condition. However digesting the vast landscape of existing studies is a challenge. The present disclosure enabled summarization of the most relevant information for researchers. Often observational study results vary due to small sample size or unknown confounding effect. The present disclosure also allows evaluation of potential bias due to cohort construction and potential confounding effect using existing databases.

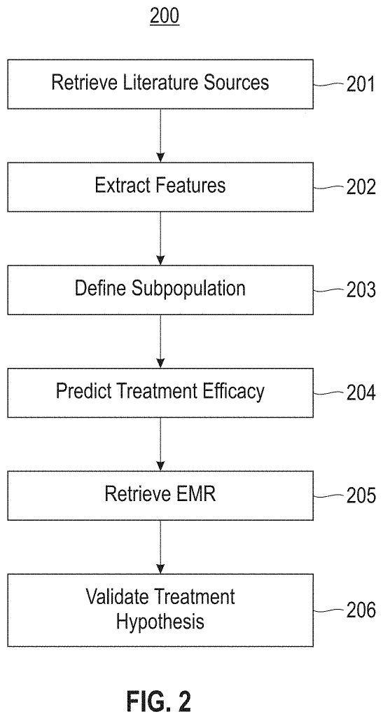

Referring now to FIG. 2, a method of treatment-effect analysis is illustrated according to embodiments of the present disclosure. At 201, a plurality of literature sources relating to a medical condition are retrieved. The plurality of literature sources comprise published observational studies. At 202, a first plurality of features is extracted from the plurality of literature sources. At 203, a subpopulation is defined based on the first plurality of features. At 204, an effectiveness of a treatment within the subpopulation is predicted based on the plurality of features. At 205, a plurality of electronic medical records is retrieved. At 206, the effectiveness of the treatment is validated against the plurality of electronic medical records.

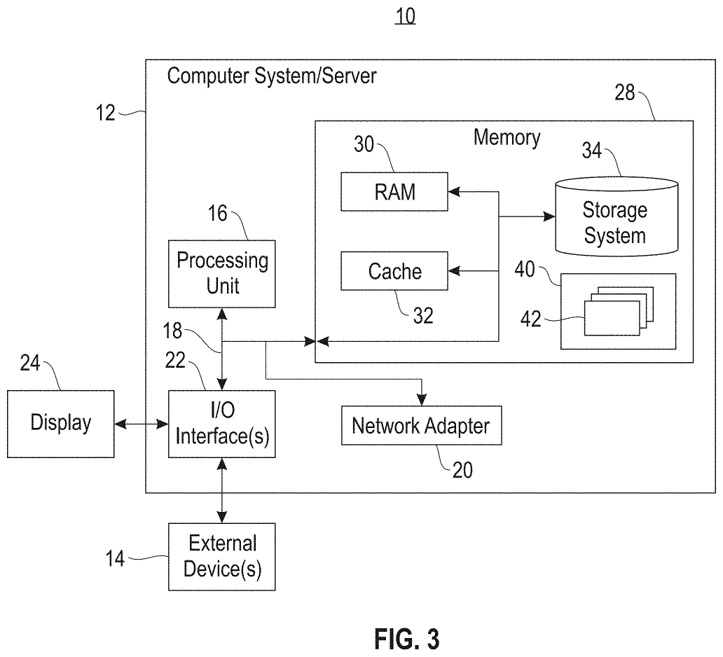

Referring now to FIG. 3, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 3, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
retrieving a plurality of literature sources relating to a medical condition, the plurality of literature sources comprising published observational studies;
extracting a first plurality of features from the plurality of literature sources;
defining a subpopulation based on the first plurality of features;
predicting an effectiveness of a treatment within the subpopulation based on the plurality of features;
retrieving a plurality of electronic medical records;
validating the effectiveness of the treatment against the plurality of electronic medical records.

2. The method of claim 1, wherein the electronic medical records comprise health data, insurance claim data, or registry data.

3. The method of claim 1, wherein the first plurality of features comprises disease definition, condition definition, outcome, standard of care, treatment, effectiveness, cohort definition, inclusion criteria, exclusion criteria, primary endpoint, secondary endpoint, potential modifier, one or more confounders, minimum sample size, or study duration.

4. The method of claim 1, wherein predicting the effectiveness comprises applying a trained model to features of the subpopulation.

5. The method of claim 4, wherein the model comprises an ensemble model.

6. The method of claim 1, wherein extracting the first plurality of features comprises applying natural language processing to the plurality of literature sources.

7. The method of claim 1, wherein validating the effectiveness of the treatment comprises determining a second plurality of features of the plurality of electronic medical records and comparing the effectiveness against those of the plurality of electronic medical records correlated with the first plurality of features.

8. A system comprising:
a computing node comprising a computer readable storage medium having program instructions embodied therewith, the program instructions executable by a processor of the computing node to cause the processor to perform a method comprising:
retrieving a plurality of literature sources relating to a medical condition, the plurality of literature sources comprising published observational studies;
extracting a first plurality of features from the plurality of literature sources;
defining a subpopulation based on the first plurality of features;
predicting an effectiveness of a treatment within the subpopulation based on the plurality of features;
retrieving a plurality of electronic medical records;
validating the effectiveness of the treatment against the plurality of electronic medical records.

9. The system of claim 8, wherein the electronic medical records comprise health data, insurance claim data, or registry data.

10. The system of claim 8, wherein the first plurality of features comprises disease definition, condition definition, outcome, standard of care, treatment, effectiveness, cohort definition, inclusion criteria, exclusion criteria, primary endpoint, secondary endpoint, potential modifier, one or more confounders, minimum sample size, or study duration.

11. The system of claim 8, wherein predicting the effectiveness comprises applying a trained model to features of the subpopulation.

12. The system of claim 11, wherein the model comprises an ensemble model.

13. The system of claim 8, wherein extracting the first plurality of features comprises applying natural language processing to the plurality of literature sources.

14. The system of claim 8, wherein validating the effectiveness of the treatment comprises determining a second plurality of features of the plurality of electronic medical records and comparing the effectiveness against those of the plurality of electronic medical records correlated with the first plurality of features.

15. A computer program product for treatment-effect analysis, the computer program product comprising a computer readable storage medium having program instructions embodied therewith, the program instructions executable by a processor to cause the processor to perform a method comprising:
retrieving a plurality of literature sources relating to a medical condition, the plurality of literature sources comprising published observational studies;
extracting a first plurality of features from the plurality of literature sources;
defining a subpopulation based on the first plurality of features;
predicting an effectiveness of a treatment within the subpopulation based on the plurality of features;
retrieving a plurality of electronic medical records;
validating the effectiveness of the treatment against the plurality of electronic medical records.

16. The computer program product of claim 15, wherein the electronic medical records comprise health data, insurance claim data, or registry data.

17. The computer program product of claim 15, wherein the first plurality of features comprises disease definition, condition definition, outcome, standard of care, treatment, effectiveness, cohort definition, inclusion criteria, exclusion criteria, primary endpoint, secondary endpoint, potential modifier, one or more confounders, minimum sample size, or study duration.

18. The computer program product of claim 15, wherein predicting the effectiveness comprises applying a trained model to features of the subpopulation.

19. The computer program product of claim 18, wherein the model comprises an ensemble model.

20. The computer program product of claim 15, wherein validating the effectiveness of the treatment comprises determining a second plurality of features of the plurality of electronic medical records and comparing the effectiveness against those of the plurality of electronic medical records correlated with the first plurality of features.
